(19) United States
(12) Patent Application Publication
(10) Pub. No.: US 2020/0156050 A1
(43) Pub. Date: May 21, 2020

(54) DEHYDROGENATION CATALYSTS FOR CONVERTING ALKYL AROMATIC COMPOUNDS SUCH AS ETHYLBENZENE

(71) Applicant: Clariant International Ltd, Muttenz (CH)

(72) Inventors: Tatiana SHVAREVA-PIEKARZ, Sacramento, CA (US); Claus G. LUGMAIR, Santa Cruz, CA (US); David LOWE, Sunnyvale, CA (US); Anthony F. VOLPE, Santa Clara, CA (US); Nobuaki KODAKARI, Hayahoshi (JP); Shinya HIRAHARA, Toyama-city Toyama (JP)

(21) Appl. No.: 16/675,395

(22) Filed: Nov. 6, 2019

Related U.S. Application Data
(60) Provisional application No. 62/769,127, filed on Nov. 19, 2018.

(51) Int. Cl. B01J 23/89; B01J 27/232; C07C 5/333

(52) U.S. Cl. CPC B01J 23/894; C07C 15/46; C07C 5/3332; B01J 27/232

(57) ABSTRACT

Inventive dehydrogenation catalysts according to multiple embodiments and alternatives contain about 60 to about 80% of iron oxide; with up to 100 ppm and in some embodiments from about 1 to about 65 ppm, of a platinum group metal or metals, being rhodium or rhodium combined with palladium; and a promoter that may include, among others, potassium and cerium; to achieve an improved ethylbenzene conversion to styrene at more favorable steam to oil ratios, including such a ratio of 0.8:1.

### CROSS-REFERENCE TO RELATED APPLICATIONS

This application claims the benefit of priority of U.S. Provisional Patent Application no. 62/769,127, filed Nov. 19, 2018, which is hereby incorporated herein by reference in its entirety.

### FIELD OF INVENTION

Current embodiments relate to production methods and use of catalysts for chemical reactions demonstrating improved activity in the production of styrene through dehydrogenation of an alkyl aromatic starting compound, i.e., ethylbenzene.

### BACKGROUND

Styrene monomer is an important petrochemical intermediate for the production of plastics, rubbers and resins, with millions of tons produced annually by catalytic dehydrogenation of ethylbenzene. Ethylbenzene dehydrogenation to styrene (C₆H₅CH═CH₂) is an equilibrium-limited and strongly endothermic process requiring high reaction temperatures and sub-atmospheric reaction pressure. The subject reaction is shown as follows and referred to elsewhere in this disclosure as Reaction (1):

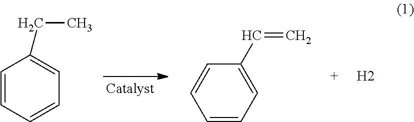

An existing process used commercially operates at about 540-620° C., and uses potassium-promoted iron oxide catalyst in the presence of superheated steam co-fed with ethylbenzene. The steam limits deposition of coke, acts as an oxidizing agent, shifts equilibrium toward the products, and supplies the heat of the reaction. Accordingly, under existing practices, an excess of steam is deemed necessary to maintain high conversion throughout the entire reactor.

However, because generating steam consumes considerable energy, it is all the more important, and even economically necessary, to effectively conduct this dehydrogenation reaction at a reduced steam feed. Accordingly, attention in the industry has turned to novel catalyst formulations that shift the reaction equilibrium toward products, reduce the energy needed to drive the dehydrogenation reaction, and present cost-effective solutions to the need for viable catalysts.

Among other factors, yield depends on the percentage conversion of reactant to product, and the selectivity of the catalyst for driving that particularly chemical conversion. Selectivity is seen in the amount of desired product, in particular styrene as distinct from other products such as benzene or toluene, produced in relation to the total amount of feedstock (ethylbenzene) that was chemically converted.

In the dehydrogenation of ethylbenzene to styrene, a favorable steam to oil ratio generally leads to a more optimal yield at lower cost. In the ratio, steam represents the numerator and can be thought of as a measure of the water (oxidative agent) and energy consumption needed for the dehydrogenation reaction to convert a specific amount of hydrocarbon, (i.e., ethylbenzene) to styrene, in which the amount of hydrocarbon (ethylbenzene) serves as the denominator for this ratio. More recent developments in this field have produced dehydrogenation catalysts operating at mild to low steam to oil ratio of about 1.2:1 or 1:1 (by weight) that provide robust yield and good selectivity. Even so, the development of such catalysts operating at an ultra-low steam to oil ratio of 0.8:1 have eluded practical application until now.

### SUMMARY OF EMBODIMENTS

Embodiments herein are directed to novel dehydrogenation catalysts demonstrating high activity and selectivity. The catalysts described herein that are within the scope of the present embodiments are effective as dehydrogenation catalysts, and particularly effective in the dehydrogenation of ethylbenzene to produce styrene. Embodiments include those in which the catalysts convert ethylbenzene to styrene through Reaction (1). In some embodiments, the inventive catalyst is characterized structurally by the addition of rhodium (Rh) in combination with palladium (Pd) at a ppm level in the catalysts' formulation, and functionally is characterized by improved activity at low temperatures. Additionally, several promoters are within the scope of present embodiments that promote catalyst activity and selectivity at lower steam to oil ratios, including without limitation potassium and cerium.

Embodiments include the inventive catalysts themselves in accordance with the compositions herein, as well as methods for their manufacture, and methods for performing dehydrogenation (including dehydrogenation of ethylbenzene to styrene) with use of the inventive catalysts.

In some embodiments directed to the preparation of a catalyst for the dehydrogenation of ethylbenzene, Pd(NO₃)₂ is combined with nitrates of other platinum group metals for the preparation of catalyst for the dehydrogenation conversion of ethylbenzene to styrene, with rhodium being one such platinum group metal and Rh(NO₃)₃ being one such nitrate. Although precursors discussed herein include Pd(NO₃)₂ for palladium and Rh(NO₃)₃ for rhodium, other known precursors for these elements could also be employed.

There is thus provided through the descriptions, teachings and embodiments herein, catalysts exhibiting improved ethylbenzene conversion, as well as methods for catalyzing this conversion. Additionally, according to at least one embodiment, this conversion with a catalyst according to multiple embodiments and alternatives herein is carried out in the presence of steam. Advantageously, the catalysts and the methods herein when used in the presence of steam achieve the conversion with a steam (numerator) to oil (denominator) ratio wherein the denominator is 1 and the numerator is about 0.8 or lower. In some embodiments, the inventive catalysts are prepared by including about 1 to about 100 ppm of at least one platinum group metal with a mixture that includes an iron compound and a promoter. In some embodiments, the content of the at least one platinum group metal in the catalyst is no greater than 65 ppm. In some embodiments, the at least one platinum group metal is only rhodium. In at least one embodiment, the at least one platinum group metal is rhodium and palladium. In some embodiments, the platinum group metal is added by blending with an iron oxide compound and at least one promoter. Alternatively, the platinum group metal is co-precipitated with the iron oxide compound. Alternatively, the platinum group metal is added as an aqueous solution, for example as metal nitrate solution.

Accordingly, the catalysts of present embodiments exhibit improved ethylbenzene conversion in the 540-570° C. temperature range compared to catalysts made only with Pd(NO₃)₂. The improvement is marked by the production of catalyst capable of operating at an ultra-low steam to oil ratio as low as 0.8:1, by conversion and selectivity comparable to the performance of commercial leading products, and by higher styrene yield in cooler reactor zones.

### BRIEF DESCRIPTION OF DRAWINGS

The drawings, figures, and descriptions provided are to be understood as illustrative, and the scope of the embodiments are not limited to the precise arrangements or data as shown in these Figures.

FIG. 1 is a graph that shows the percentage of ethylbenzene converted in relation to reaction zone temperature for catalysts evaluated in accordance with Reaction (1).

FIG. 2 is a graph that shows selectivity for styrene conversion relative to ethylbenzene conversion for the same catalysts depicted in FIG. 1.

### MULTIPLE EMBODIMENTS AND ALTERNATIVES

In some embodiments, catalysts demonstrating improved product yields associated with Reaction (1) comprise rhodium and palladium.

In some embodiments, the catalysts comprise an iron compound which is iron oxide, as Fe₂O₃, and at least one platinum group metal, being formed from nitrates of platinum group metals, including particularly Rh(NO₃)₃ and in some embodiments Pd(NO₃)₂. In some embodiments, the catalysts include both palladium and rhodium, with various binders, promoters, and modifiers as described herein. Catalysts in accordance with the present embodiments exhibit improved ethylbenzene conversion in the 540-570° C. temperature range compared to catalysts made only with Pd(NO₃)₂. Additionally, catalysts in accordance with present embodiments operate to achieve desirable yields at ultra-low steam to oil ratios, e.g., 0.8:1.

In some embodiments, the inventive catalysts contain about 60% to about 80% by weight iron oxide, which in some embodiments is Fe₂O₃, but other forms of iron oxide can be utilized as known in the art; from about 6% to about 25% by weight of a promoter comprising an alkali or transition metal, including without limitation potassium or cerium or both, with potassium source materials in a form as potassium carbonate or potassium oxide and cerium source materials in a form as cerium oxide; and from about 1 to about 100 parts per million (ppm) of one or more platinum group metals, including without limitation, rhodium or a combination of rhodium and palladium. The weight percentages described herein are based on the total catalyst weight. In some embodiments, certain binding agents as known in the art are added to the catalyst formulation for enhanced physical strength, including but not limited to perlite, aluminates, and silicates. Additional materials of the kind and weight percentages set forth in the examples may also be used within in accordance with present embodiments.

In some embodiments, the catalysts are prepared by blending the ingredients together with sufficient water to make a moist extrudable mixture, which is then extruded to produce extrudates of a particular shape and dimension as desired and appropriate for use, e.g., cylindrical pellets with a diameter of about 3 mm. The extrudates are then heat treated under conventional calcining conditions, which can range from about 500 to 1200° C. and typically will be in a range from about 600 to 1000° C. Calcination is conducted under conditions as selected by a producer as known in the art, for example under static conditions in a furnace, or under dynamic conditions such as a rotary kiln, with temperatures and residence times being determined for each individual type of catalyst according to known processes in the art.

In some embodiments, rather than blending the platinum group metal(s) with the other ingredients, such metals (e.g., rhodium or rhodium combined with palladium) are co-precipitated with iron oxide before the blending step. Alternatively, one or more platinum group metals are impregnated onto the surface of the finished catalyst by drying and re-calcining at a temperature adequate to remove water and decompose impregnated salts. Still yet another alternative would be to add such a metal or metals in the form of an aqueous solution of appropriate salts, preferably nitrates, directly to the catalyst during blending, and immediately prior to extrudation and pelletization.

While exemplary techniques for producing the catalysts are described herein, generally catalysts of the present embodiments can be synthesized using techniques known in the art, including without limitation precipitation; impregnation; incipient wetness impregnation upon mesoporous or microporous supports; spray drying; solvent evaporation; and other methods known in the art and currently existing or later developed.

In some embodiments, one or more catalysts according to the present teachings catalyze the conversion of ethylbenzene to styrene, the steps of which comprise contacting ethylbenzene reactants with a dehydrogenation catalyst comprising about 60% to about 80% of an iron compound, which can be iron oxide, about 20% to about 30% of a promoter, and about 1 to about 100 ppm of a platinum group metal, and isolating styrene products.

In view of the teachings contained herein, the following examples identify some of the ways in which the present embodiments can be practiced and the results obtained. These examples are not limiting, but rather are meant to illustrate further the teachings herein.

### EXAMPLES 1-4

A plurality of dehydrogenation catalysts were formed from a starting mixture of 37.792 g of Fe₂O₃, 11.168 g of K₂CO₃, 6.940 g of Ce₂(CO₃)₃, 3.440 g of CeOHCO3, 0.327 g of MoO₃, 0.720 g of Ca(OH)₂, 1.820 g Na₂CO₃, 0.16 g of Y₂O₃ and 0.4 g of methyl cellulose as an extrusion aid. For each example, the constituents were mixed in a high speed blade mixer for 1 min until a dry homogeneous mixture was produced. At the point of the dry homogeneous mixture:

With Example 1, a 0.72 ml portion of a 0.01082 M Rh(NO₃)₃ solution was added to the homogenous contents of the mixer, followed by the addition of 0.8 g of extrusion oil, and 4 g water. At this point, the mixture was blended for an additional 1 min, to form a moist paste.

With Example 2, a 0.24 ml portion of 0.01082 M Rh(NO₃)₃ was mixed with 0.72 ml portion of a 0.01167 M Pd(NO₃)₂ solution and then added to the homogenous contents of the mixer followed by the addition of 0.8 g of extrusion oil, and 3.76 g water. At this point, the mixture was blended for an additional 1 min, to form a moist paste.

With Example 3, a 0.72 ml portion of 0.01082 M Rh(NO₃)₃ was mixed with 0.72 ml portion of a 0.01167 M Pd(NO₃)₂ solution and then added to the homogenous contents of the mixer followed by the addition of 0.8 g of extrusion oil, and 3.28 g water. At this point, the mixture was blended for an additional 1 min, to form a moist paste.

With Example 4, a 2.4 ml portion of 0.01082 M Rh(NO₃)₃ was mixed with a 0.72 ml portion of a 0. 01167 M Pd(NO₃)₂ solution was added to the homogenous contents of the mixer followed by the addition of an 0.8 g of extrusion oil, and 1.75 g water. At this point, the mixture was blended for an additional 1 min, to form a moist paste.

With each of Examples 1-4, at the point of the moist paste, this paste was then extruded in a piston extruder, transferred to a mesh basket and dried at 120° C. for half an hour, and calcined at 900° C. in air for two hours to form a promoted iron oxide catalyst.

### COMPARATIVE EXAMPLE EXAMPLE 5, PALLADIUM

Prior to the present application, addition of palladium at ppm levels was used to improve catalytic activity, presumably by increasing the rate of iron oxide surface regeneration. Aqueous solutions of palladium nitrate have been used as an effective precursor of palladium.

As a comparative, then, as with Examples 1-4, for Example 5 a catalyst was formed and used as a comparative example from a starting mixture of 37.79 g of Fe₂O₃, 11.17 g of K₂CO₃, 6.94 g of Ce₂(CO₃)₃, 3.44 g of CeOHCO₃, 0.37 g of MoO₃, 0.72 g of Ca(OH)₂, 1.82 g Na₂CO₃, 0.16 g of Y₂O₃ and 0.4 g of methyl cellulose. The constituents were mixed in a high speed blade mixer for 1 min until a dry homogeneous mixture was produced. At the point of the homogenous mixture, a 0.72 ml portion of a 0.01167 M Pd(NO₃)₂ solution was added to the mixer followed by the addition of an 0.8 g of extrusion oil, and 4 g water, resulting in 15 ppm of palladium in the catalyst. The mixture was blended for an additional 1 min, to form a moist paste. The paste was then extruded in a piston extruder, transferred to a mesh basket and dried at 120° C. for half an hour and calcined at 900° C. in air for two hours.

Table 1 summarizes the differences among Examples 1-4, of which Example 5 is comparative. In each example, the materials forming the dry homogenous mixture were the same, i.e., 37.792 g of Fe₂O₃, 11.168 g of K₂CO₃, 6.940 g of Ce₂(CO₃)₃, 3.440 g of CeOHCO₃, 0.327 g of MoO₃, 0.720 g of Ca(OH)₂, 1.820 g Na₂CO₃, 0.16 g of Y₂O₃ and 0.4 g of methyl cellulose.

TABLE 1

| | Platinum | Aqueous solution | |
|---|---|---|---|
| | group metal | added to homogenous | Added |
| | concentration | mixture | constituents |
| Example 1 | 15 ppm Rh | 0.72 ml portion of | 0.8 g of extrusion |
| | | 0.01082M Rh(NO₃)₃ | oil, and 4 g water |
| Example 2 | 5 ppm Rh, | 0.24 ml portion of | 0.8 g extrusion |
| | 15 ppm Pd | 0.01082M Rh(NO₃)₃ | oil and 3.76 g water |
| | | mixed with | |
| | | 0.72 ml portion of | |
| | | 0.01167M Pd(NO₃)₂ | |
| | | solution | |
| Example 3 | 15 ppm Rh, | 0.72 ml portion of | 0.8 g extrusion |
| | 15 ppm Pd | 0.01082M Rh(NO₃)₃ | oil and 1.75 g water |
| | | mixed with | |
| | | 0.72 ml portion of | |
| | | 0.01167M Pd(NO₃)₂ | |
| | | solution | |
| Example 4 | 50 ppm Rh, | 2.4 ml portion of | 0.8 g extrusion |
| | 15 ppm Pd | 0.01082M Rh(NO₃)₃ | oil and 4 g water |
| | | mixed with | |
| | | 0.72 ml portion of | |
| | | 0.01167M Pd(NO₃)₂ | |
| | | solution | |
| Example 5 | 15 ppm Pd | 0.72 ml portion of | 0.8 g extrusion |
| | | 0.01167M Pd(NO₃)₂ | oil and 4 g water |
| | | solution | |

Dehydrogenation of Ethylbenzene using Examples 1-5

Each promoted iron oxide catalyst of Examples 1-4, being within the scope of present embodiments, was employed for the dehydrogenation of ethylbenzene in a fixed bed reactor. Likewise, the catalyst of the comparative example (Example 5) was employed for the dehydrogenation of ethylbenzene in a fixed bed reactor.

Ethylbenzene was co-fed with steam at 0.8:1 steam to oil ratio. For each of Examples 1-5, 0.340 g of catalyst were loaded between two layers of alumina in a fixed bed reactor. After heating the reactor in N₂ flow to 600° C. and stabilization in N₂/H₂O for 30 min, the feed with composition 6.12 mol % ethylbenzene, 28.88 mol % H₂O, and 65 mol % N₂ was continuously flowed through the bed to achieve liquid hourly space velocity of 1 h⁻¹. The system was then heated to 620° C. The product stream was analyzed intermittently during a stabilization period of about 300 hours, and measurements were taken at 620° C., 600° C., 570° C. and 540° C. with an equilibration period of 12 hours after each temperature change. Average conversion was calculated for conversion at 620° C., 600° C., and 570° C. T₄₀ is a temperature of the reaction zone corresponding to 40% conversion of ethylbenzene to styrene. T₄₀ is a temperature of the reaction zone corresponding to 60% conversion of ethylbenzene to styrene. S₄₀ is selectivity at 40% conversion of ethylbenzene to styrene. S₆₀ is selectivity at 60% conversion of ethylbenzene to styrene.

### Results and Data

Table 2 shows the performance of Examples 1-4 as well as the Comparative Example for ethylbenzene dehydrogenation according to Reaction (1), in a fixed-bed reactor with a steam to oil ratio of 0.8:1.

TABLE 2

| | | | | | | | Con- |
|---|---|---|---|---|---|---|---|
| | Platinum | Average | | | | | version |
| | group | con- | | | | | at |
| | metal | version, | T₄₀, | S₄₀, | T₆₀, | S₆₀ | 570° C., |
| Example | concentration | % | ° C. | % | ° C. | % | % |
| Example 1 | 15 ppm Rh | 61.23 | 563 | 98.90 | 594 | 98.30 | 44.69 |
| Example 2 | 5 ppm Rh, | 64.36 | 555 | 98.88 | 588 | 98.30 | 48.92 |
| | 15 ppm Pd | | | | | | |
| Example 3 | 15 ppm Rh, | 62.56 | 556 | 99.05 | 590 | 98.52 | 47.92 |
| | 15 ppm Pd | | | | | | |
| Example 4 | 50 ppm Rh, | 57.47 | 566 | 98.98 | 600 | 98.45 | 42.12 |
| | 15 ppm Pd | | | | | | |
| Example 5 | 15 ppm Pd | 63.42 | 560 | 98.85 | 590 | 98.25 | 46.45 |

From Table 2, the temperature at which 60% of the ethylbenzene reactants undergo a conversion is not greater than 600° C. Multiplying by the lowest S₆₀ value for Examples 1-4 (selectivity for conversion to styrene), 60%×0.983 is 59.0%. Thus, with catalyst of Examples 1-4, at a temperature no higher than 600° C., at least 59% of the ethylbenzene is converted to styrene.

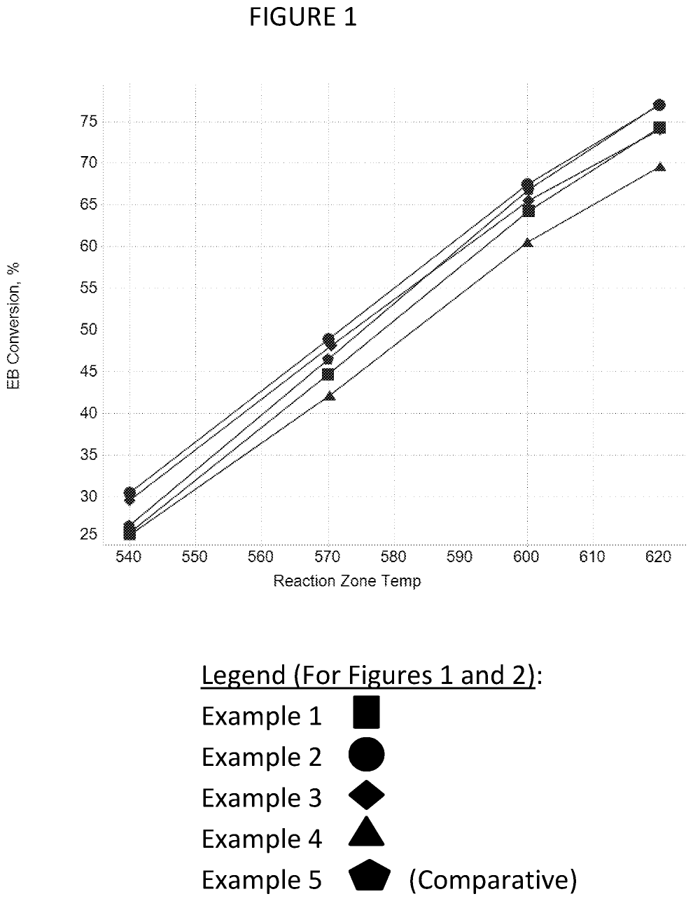

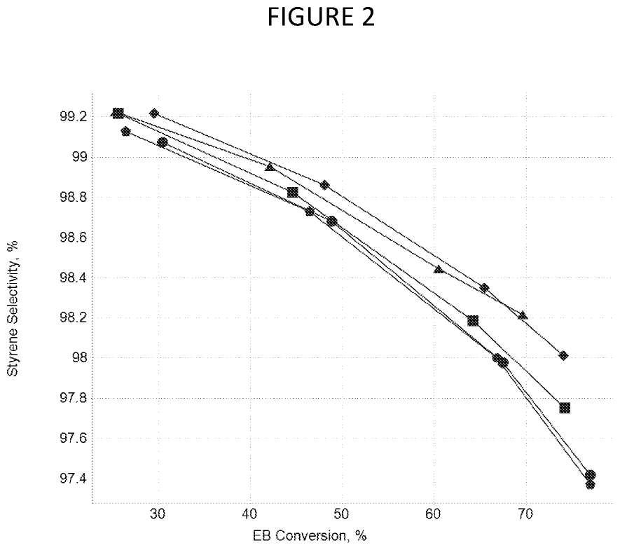

Likewise, FIGS. 1-2 graphically depict the performance of Examples 1-4 as well as the Comparative Example for ethylbenzene dehydrogenation according to Reaction (1), in a fixed-bed reactor with a steam to oil ratio of 0.8:1.

Still further, Table 3 (below) lists additional data regarding the catalysts associated with Examples 1-4 as well as the Comparative Example (Example 5).

TABLE 3

| | Average | | | | | | Crush |
|---|---|---|---|---|---|---|---|
| Catalyst | conversion | T₄₀, | S₄₀, | T₆₀, | S₆₀ | Rh (ppm)/ | strength |
| (Ex.) | (%) | ° C. | ° C. | ° C. | ° C. | Pd (ppm) | (N/mm) |
| 1 | 61.23 | 563 | 98.90 | 594 | 98.30 | 15/0 | 32.4 |
| | (2 runs) | | | | | | |
| 2 | 64.36 | 555 | 98.88 | 588 | 98.30 | 5/15 | 27.7 |
| | (2 runs) | | | | | | |
| 3 | 62.55 | 556 | 99.04 | 590 | 98.52 | 15/15 | 33.4 |
| | (2 runs) | | | | | | |
| 4 | 57.47 | 566 | 98.98 | 600 | 98.45 | 50/15 | 33.0 |
| 5 | 63.42 | 560 | 98.85 | 590 | 98.25 | 0/15 | 33.1 |

In view of the above, Examples 1-4 contained rhodium (either with palladium or as the only platinum group metal) and exhibited higher selectivity for conversion to styrene than Example 5 with only palladium. In other words, of the ethylbenzene converted with the Examples 1-4 catalysts, the percentage of styrene as the conversion product was greater than for the comparative example (Example 5). Accordingly, the inventive catalysts demonstrate desirable chemical and physical properties for the conversion of ethylbenzene, particularly its conversion to styrene, with physical strength comparable to previously used catalysts.

It will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways. Also, it is to be understood that words and phrases used herein are for the purpose of description and should not be regarded as limiting. The use herein of such words and phrases as “including,” “such as,” “comprising,” “e.g.,” “containing,” or “having” and variations of those words is meant to encompass the items listed thereafter, and equivalents of those, as well as additional items.

Accordingly, the foregoing descriptions of embodiments and alternatives are meant to illustrate, rather than to serve as limits on the scope of what has been disclosed herein. The descriptions herein are not meant to limit the understanding of the embodiments to the precise forms disclosed. It will be understood by those having ordinary skill in the art that modifications and variations of these embodiments are reasonably possible in light of the above teachings and descriptions.

1. A dehydrogenation catalyst comprising about 60% to about 80% of an iron compound, about 20% to about 30% of a promoter, and about 1 to about 100 ppm of a platinum group metal.

2. The dehydrogenation catalyst of claim 1, wherein the iron compound comprises iron oxide.

3. The dehydrogenation catalyst of claim 1, wherein the platinum group metal comprises rhodium.

4. The dehydrogenation catalyst of claim 3, wherein the platinum group metal comprises only rhodium and the amount of platinum group metal does not exceed 65 ppm.

5. The dehydrogenation catalyst of claim 1, wherein the promoter comprises a potassium compound.

6. The dehydrogenation catalyst of claim 5, wherein the promoter source comprises potassium carbonate.

7. The dehydrogenation catalyst of claim 1, wherein the promoter comprises a cerium compound.

8. The dehydrogenation catalyst of claim 7, wherein the promoter source comprises cerium oxide.

9. A method of catalyzing the conversion of ethylbenzene to styrene, comprising contacting ethylbenzene reactants with the dehydrogenation catalyst of claim 1 in the presence of steam, and isolating styrene products.

10. The method of claim 9, wherein a steam to oil ratio is no greater than 0.8:1.

11. The method of claim 9, wherein at least 59.0% of ethylbenzene is converted to styrene at a temperature no greater than 600° C.

12. The method of claim 9, wherein the iron compound comprises iron oxide.

13. The method of claim 9, wherein the platinum group metal comprises rhodium.

14. The method of claim 13, wherein the platinum group metal comprises only rhodium.

15. The method of claim 9, wherein the promoter comprises a potassium compound.

16. The method of claim 9, wherein the promoter comprises a cerium compound.
